(19) United States
(12) Patent Application Publication
(10) Pub. No.: US 2016/0313706 A1
(43) Pub. Date: Oct. 27, 2016

(54) Wrist Watch and Operation Method for Application Program Thereof

(71) Applicant: HUAQIN TELECOM TECHNOLOGY CO., LTD., Shanghai (CN)

(72) Inventors: Lei San, Shanghai (CN); Guowang Jiang, Shanghai (CN)

(73) Assignee: Huaqin Telecom Technology Co., Ltd., Shanghai (CN)

(21) Appl. No.: 15/104,902

(22) PCT Filed: Nov. 18, 2014
(86) PCT No.: PCT/CN2014/091355

§ 371 Date: Jun. 15, 2016

(30) Foreign Application Priority Data
Dec. 24, 2013 (CN) 201310724253.9
Dec. 24, 2013 (CN) 201320861196.4

(51) Int. Cl. G04G 21/02; G06F 3/041; G06F 3/01; A61B 5/024; A61B 5/00

(52) U.S. Cl. CPC G04G 21/025; A61B 5/02438; A61B 5/681; G06F 3/017; G06F 3/041; A61B 2562/0247; G06F 2203/04105

(57) ABSTRACT

A wrist watch comprising a watch dial, a watch band, a memory and a process wherein at least one pressure sensor is provided on the watch dial and/or the watch band, and the memory stores pressure events, application programs and the corresponding relationship between each of the pressure events and an application program. Each pressure sensor detects the applied pressure in real time and sends a pressure signal to the processor. The processor, queries and obtains a pressure event which matches the pressure signal from the memory, and controls the wrist watch to operate the application program corresponding to the obtained pressure event. A user can start the application program the wrist watch only via a hand action without operating a touchscreen manually, thereby providing a brand-new manner of operating a wrist watch. Also provided is an operation method for an application program of a wrist watch.

The present application requests priorities of Chinese patent application CN201310724253.9 filed on Dec. 24, 2013 and Chinese patent application CN201320861196.4 filed on Dec. 24, 2013. The above applications are entirely incorporated by reference.

### FIELD OF INVENTION

The present invention relates to a wrist watch and an operation method for application program thereof, especially to a wrist watch utilizing pressure sensor to implement application program and an operation method for the application program utilizing said wrist watch.

### PRIOR ARTS

With the development of technology, various intelligent wrist watches are available on the market. These intelligent wrist watches not only have the function of displaying time, but also are integrated with various application programs such as camera and Bluetooth etc., which is equivalent to an intelligent mobile terminal with several functions. Furthermore, these intelligent wrist watches further includes touchscreen touched by user to start the application program, which follows the conventional touch setting to the intelligent phone on the market.

However, the setting of touch operation of the current intelligent wrist watch is still conventional, which requires user to use the other hand to perform touch operation. After a user wears the wrist watch, he/she cannot operate touchscreen by one hand, thus cannot start corresponding application program. This disadvantage limits the user's operation greatly and influences user's experience.

### CONTENT OF THE PRESENT INVENTION

The technical problem to be solved by the present invention is to overcome the defect that the user requires to operate the touchscreen to start corresponding application program in the prior art, which limits user's operation greatly and influences user's experience. To solve this technical problem, the present invention provides a wrist watch utilizing pressure sensor to implement application program and an operation method for the application program utilizing said wrist watch.

The present invention solves the above technical problem by the following technical solutions:

The present invention provides a wrist watch comprising as watch dial and a watch band, wherein at least one pressure sensor is provided on the watch dial and/or the watch band, and the wrist watch also comprises a memory and a processor;

the memory is configured to store at least one pressure event, at least one application program of the wrist watch and the corresponding relationship between each pressure event and an application program, and the pressure event comprises the duration time of a pressure and the range of a pressure value;

each pressure sensor is configured to detect the applied pressure in real time and send a pressure signal to the processor, and the pressure signal comprises the duration time of a pressure and a pressure value;

the processor is configured to query and obtain a pressure event which matches the pressure signal from the memory, and control the wrist watch to operate the application program corresponding to the obtained pressure event.

Preferably, each pressure sensor has a unique identification code, and both the pressure event and the pressure signal include identification code of the pressure sensor.

When a user wears the wrist watch and cannot operate the touchscreen of the wrist watch by one hand, in the present invention the hand (wrist and palm, etc.) action of the user is sensed by the pressure sensor; and the sensed pressure signal is delivered by the pressure sensor; and the pressure signal and the pressure event stored in the memory are matched; when the identification code, the duration time of a pressure and the range of a pressure value of the pressure signal match those of the pressure event, the processor queries and obtains the pressure event which matches the pressure signal from the memory, and then starts the corresponding application program. Thereby, the user does not need to operate a touchscreen manually, that is to say, he/she can start the application program in the wrist watch by merely performing different hand actions with the hand wearing the wrist watch and applying different pressures. In this manner, a brand-new manner of operating a wrist watch is provided, which enriches the user's operation greatly and enhances the user's experience.

Preferably, the processor comprises a pressure event generation module, which includes a detect unit, a counter whose initial value is zero and a control unit;

With regard to the pressure signal sent by each pressure sensor, the detect unit is configured to identify the identification code of the pressure sensor, and detect whether the duration time of a pressure exceeds a first threshold and whether the pressure value exceeds a second threshold, if the results of both detection are “yes”, the counter is controlled to add 1 to count value, and when the count value of the counter equals to a third threshold, the control unit is activated to generate a pressure event from the pressure signal, and stores the pressure event in the memory.

The control unit is further configured to display at least one application program on the display of the wrist watch, and it is configured to pair the generated pressure event with the selected application program after receiving an instruction of selecting an application program.

The pressure event generation module can generate specific pressure event according to the pressure signal meeting specific condition from the user, which enables the user to set the corresponding relationship between the pressure event and the application program. A plurality of pressures continuously applied by the user should meet the same specific condition substantially, thus reducing the possibility of misoperation greatly.)

Preferably, when the pressure sensor and the processor are wired, the detect unit is configured to identify the pressure sensor according to pins on the line; when the pressure sensor and the processor are wirelessly connected, the pressure signal sent by the pressure sensor contains a field representing the identification code, the detect unit is configured to identify the pressure sensor according to the field representing the identification code.

Preferably, when the pressure sensor locates at the pulse position of human body, the pressure sensor is configured to detect the pressure generated by the pulse of human body and send a pulse pressure signal to the processor, and the processor further includes a filter module for filtering the pulse pressure signal from the pressure signal sent by the pressure sensor.)

If the pressure sensor is disposed at the pulse position of hand, when no pressure generated from a hand action of the user is received, the pressure sensor can detect pulse pressure signal generated by the pulse of human body, and the processor thus filters the pulse pressure signal by the filter module to eliminate interference to the pressure sensor due to the pulse of human body.

Preferably, each pressure sensor detects the pressure generated by a hand action of human body. In this present invention, most of the pressures are generated by hand actions of the hand wearing the wrist watch, such hand actions include rotation of wrist, upward and downward swing of the wrist, and clench and release of the palm etc.

Preferably, at least one pressure sensor is disposed on backside of the watch dial and/or the watch band.

The present invention further provides an operation method for application program of a wrist watch, wherein the operation method implanted by utilizing the above mentioned, wrist watch includes the following steps:

S1, the memory stores at least one pressure event, at least one application program of the wrist watch and the corresponding relationship between each pressure event and an application program, and the pressure event comprises duration time of a pressure and range of a pressure value;

S2, each pressure sensor detects the applied pressure in real time and sends a pressure signal to the processor, and the pressure signal includes duration time of a pressure and a pressure value;

S3, the processor queries and obtains a pressure event which matches the pressure signal from the memory, and controls the wrist watch to operate the application program corresponding to the obtained pressure event.

Preferably, each pressure sensor has a unique identification code, and both the pressure event and the pressure signal include identification code of the pressure sensor.

Preferably, the processor comprises a pressure event generation module, which includes a detect unit, a counter whose initial value is zero and a control unit; and prior to step S1, said method further comprises a pressure event generation step;

With regard to the pressure signal sent by each pressure sensor, the detect unit identifies the identification code of the pressure sensor, and detects whether the duration time of a pressure exceeds a first threshold and whether the pressure value exceeds a second threshold, if the results of both detection are “yes”, the counter is controlled to add 1 to count value, and when the count value of the counter equals to a third threshold, the control unit is activated to generate a pressure event from the pressure signal, and stores the pressure event in the memory;

the control unit further displays at least one application program on the display of the wrist watch, and it pairs the generated pressure event with the selected application program after receiving an instruction of selecting an application program.

Preferably, in the pressure event generation step, when the pressure sensor and the processor are wired, the detect unit is configured to identify the pressure sensor according to pins on the line; when the pressure sensor and the processor are wirelessly connected, the pressure signal sent by the pressure sensor contains a field representing the identification code, the detect unit is configured to identify the pressure sensor according to the field representing the identification code.

Preferably, in the operation method, when the pressure sensor locates at the pulse position of human body, the pressure sensor detects the pressure generated by the pulse of human body and sends a pulse pressure signal to the processor, and the processor further includes a filter module for filtering the pulse pressure signal from the pressure signal sent by the pressure sensor.

Preferably, in step S2, each pressure sensor detects the pressure generated by a hand action of human body.

The positive effect of the present invention lies in: in the present invention the a hand action of the user is sensed by the pressure sensor, the sensed pressure signal is delivered by the pressure sensor, the pressure signal and the pressure event stored in the memory are matched, and the application program corresponding to the pressure event is started. Thereby, the user does not need to operate a touchscreen manually, that is to say, he/she can start the application program in the wrist watch by merely performing different hand actions with the hand wearing the wrist watch, and applying different pressures. In this manner, a brand-new manner of operating a wrist watch is provided, which enriches the user's operation greatly and enhances the user's experience.

### BRIEF DESCRIPTION OF THE DRAWINGS

FIG. 1 is a structural diagram of the wrist watch according to embodiment 1 of the present invention.

FIG. 2 is a flow chart representing the operation method for application program of the wrist watch of embodiment 1 of the present invention.

FIG. 3 is a structural diagram of the wrist watch according to embodiment 2 of the present invention.

FIG. 4 is a flow chart representing the operation method for application program of the wrist watch of embodiment 2 of the present invention.

FIG. 5 is a structural diagram of the wrist watch according to embodiment 3 of the present invention.

### DETAILED DESCRIPTION OF THE PREFERRED EMBODIMENT

### Embodiment 1

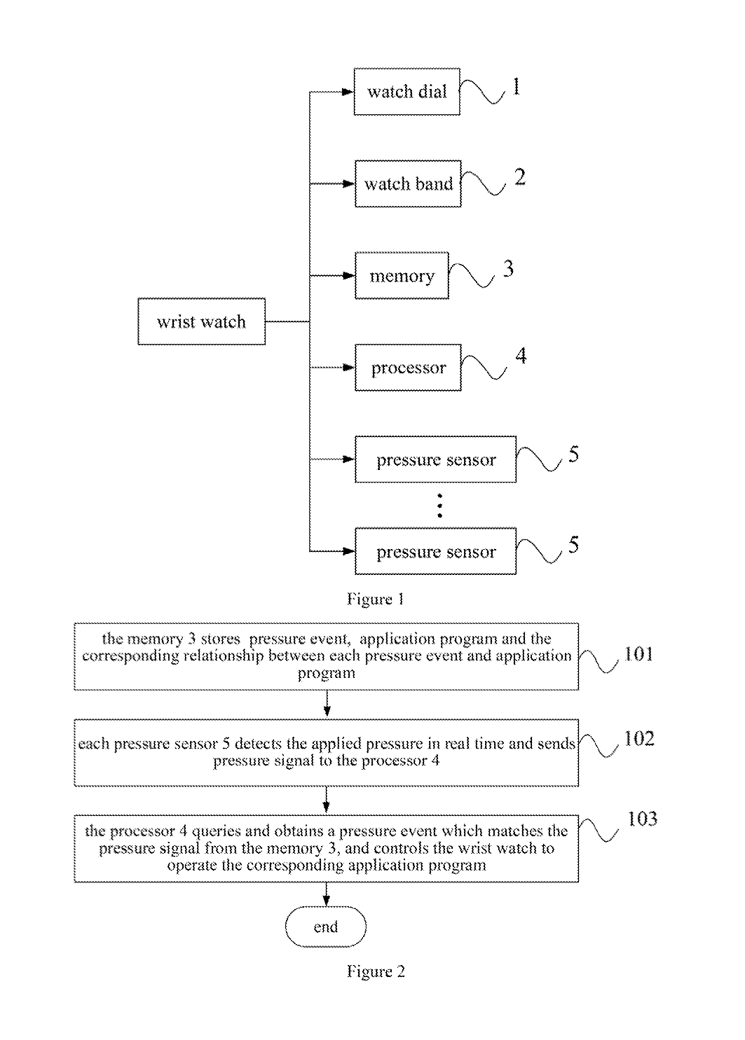

As shown in FIG. 1, the wrist watch of the present embodiment includes a watch dial 1, a watch band 2, a memory 3, a processor 4 and at least one pressure sensor 5 provided, on the watch dial 1 and/or the watch band 2, and each pressure sensor 5 has a unique identification code.

The number of the pressure sensor 5 can be set according to actual needs, and the position of each pressure sensor 5 can also be set. For example, if two pressure sensors are provided, one pressure sensor can be disposed on the inner surface of the watch dial 1, the other pressure sensor can be disposed inside the watch hand 2 at a position facing the watch dial 1 or at a position forming an angle such as 90 degrees with respect to the watch dial. Due to enrichment of the blood vessels and muscles inside the wrist of human body, when the user wears the wrist watch of the present embodiment and performs a hand action such as release or clench of palm, more pressure will be applied to the pressure sensor 5 to facilitate collecting pressure signal. When the pressure sensor 5 is disposed inside the watch dial 1, it can be encapsulated on the motherboard in the watch dial 1 by surface encapsulation technology. Of course, three or four pressure sensors can be provided, and their positions can be set according to the above arrangement.

The memory 3 pre-stores at least one pressure event and at least one application program of the wrist watch such as camera and Bluetooth etc., wherein each pressure event is corresponding to an application program, the pressure event comprises the identification code of pressure sensor 5, the duration time of a pressure and the range of a pressure value. With respect to the pressure sensors 5 with the same identification code, if the duration time of the pressure thereof and the pressure value thereof are both different, they represent different pressure events. Of course, with respect to pressure sensors 5 with different identification codes, they also represent different pressure events.

When the user wears wrist watch and controls the hand to perform actions such as rotation of wrist, upward and downward swing of the wrist and clench or release of the palm, different actions will apply different pressures to different pressure sensors 5. The pressure sensor 5 can detect the pressure in real time, then generate a pressure signal and send it to the processor 4. The information carried by the pressure signal includes identification code of the pressure sensor 5, duration time of a pressure and the range of a pressure value.

After the processor 4 receives the pressure signal, it will traverse the pressure events stored in the memory 3, query and obtain a pressure event which matches the pressure signal. Said match means that the identification codes of the pressure sensor 5 in the pressure signal and the pressure event are identical, and the difference fluctuations of the pressure duration time and the pressure value fall into a preset threshold range, wherein the specific fluctuation threshold can be set according to actual needs.

After obtaining the pressure event corresponding to the received pressure signal, the processor 4 starts the application program corresponding to the obtained pressure event such as camera. Thereby, the pressures are applied by merely performing different hand actions, and then the wrist watch is controlled to operate the corresponding application programs. In this manner, a brand-new manner of operating a wrist watch is provided, which enriches the user's operation greatly and enhances the user's experience.

As shown in FIG. 2, the operation method for application program of a wrist watch implemented by utilizing the wrist watch of the present embodiment includes the following steps:

Step 101, the memory 3 stores at least one pressure event, at least one application program of the wrist watch and the corresponding relationship between each pressure event and an application program.

Step 102, each pressure sensor 5 detects the applied pressure in real time and sends a pressure signal to the processor 4.

Step 103, the processor 4 queries and obtains a pressure event which matches the pressure signal from the memory 3, and controls the wrist watch to operate the application program corresponding to the obtained pressure event, then the procedure ends.

### Embodiment 2

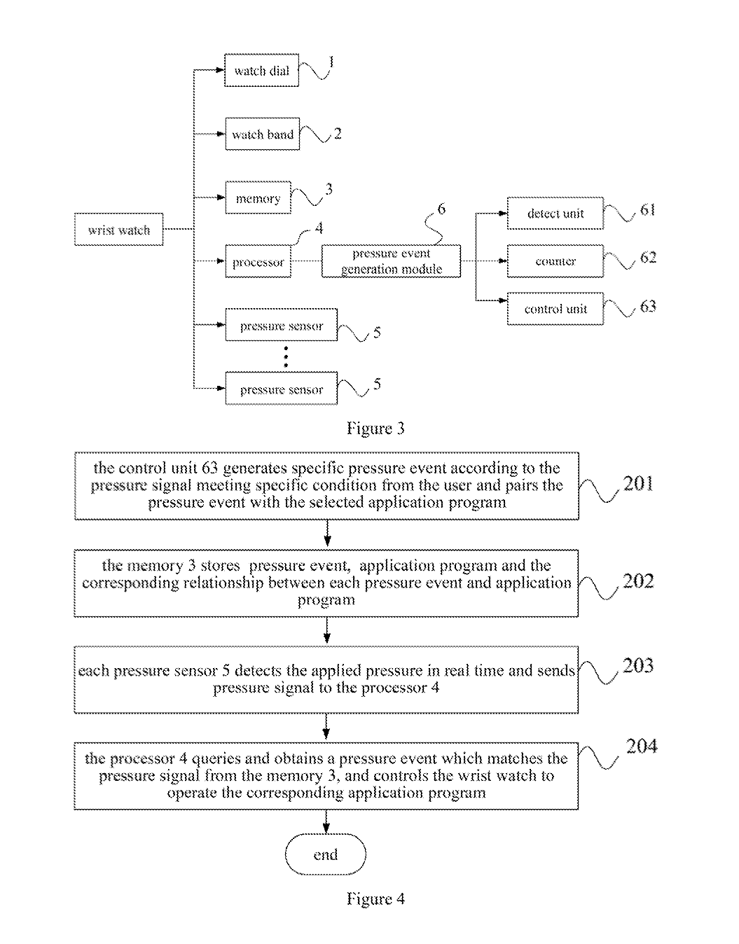

As shown in FIG. 3, in the wrist watch of the present embodiment, the processor 4 further comprises a pressure event generation module 6, which includes a detect unit, a counter 62 whose initial value is zero and a control unit 63. The difference between the present embodiment and embodiment 1 lies in: in the present embodiment, the pressure event generation module 6 can generate specific pressure event, according to the pressure sent from the user to the pressure sensor 5.

After receiving the pressure from the user, each pressure sensor 5 will send a pressure signal, and the detect unit 61 can identify the identification code of the pressure sensor 5 according to the pressure signal, and detect whether the duration time of the pressure exceeds a first threshold and whether the pressure value exceeds a second threshold (the first threshold and the second threshold can be set according to actual needs, for different pressure sensors 5, the corresponding first threshold and second threshold may be different), if the results of both detection are “yes”, the counter 62 is controlled to add to count value.

Once the user applies the pressure meeting the above requirements, the count value of the counter 62 will be added to 1. When the count value of the counter 62 increases to the third threshold (i.e. applying the required pressure to the same pressure sensor 5 several times), the control unit 63 is activated to generate a pressure event from the pressure signal, and stores the pressure event in the memory 3. The information carried by the pressure event includes the identification code of the pressure sensor 5, the duration time of a pressure and the range of a pressure value.

The control unit 63 is further configured to display at least one application program on the display of the wrist watch, and the user can select one application program by the touchscreen. The control unit 63 can pair the generated pressure event with the selected application program.

In this manner, in the present embodiment the specific pressure event can be generated by the pressure event generation module 6 according to the pressure signal sent from the user by his/her hand action, and the user can set the corresponding relationship between each pressure event and the application program. After setting the corresponding relationship between each pressure event and the application program and storing this relationship in the memory 3, the user applies pressure to the pressure sensor 5 by hand actions including rotation of wrist, upward and downward swing of the wrist and clench and release of the palm, and then the wrist watch is controlled to operate the application program with the same processing mode and specific procedure in embodiment 1, the details thereof omitted.

In the present embodiment, the pressure sensor 5 and the processor 4 can be wired or wirelessly connected. When the pressure sensor 5 and the processor 4 are wired, the detect unit 61 can identify the identification code of the pressure sensor 5 according to different pins on the line; when the pressure sensor 5 and the processor 4 are wirelessly connected, the pressure signal sent by the pressure sensor 5 includes a field representing the identification code, and the detect unit 61 can identify the identification code of the pressure sensor 5 according to the field.

As shown in FIG. 4, the operation method for application program of a wrist watch implemented by utilizing the wrist watch of the present embodiment includes the following steps:

Step 201, with regard to the pressure signal sent by each pressure sensor 5, the detect unit 61 identifies the identification code of the pressure sensor 5, and detects whether the duration time of a pressure exceeds a first threshold and whether the pressure value exceeds a second threshold, if the results of both detection are “yes”, the counter 62 is controlled to add 1 to count value, when the count value of the counter 62 equals to a third threshold, the control unit 63 is activated to generate a pressure event from the pressure signal, and stores the pressure event in the memory 3;

the control unit 63 further displays at least one application program on the display of the wrist watch, and it pairs the generated pressure event with the selected application program after receiving an instruction of selecting an application program.

Step 202, the memory 3 stores at least one pressure event, at least one application program of the wrist watch and the corresponding relationship between each pressure event and an application program.

Step 203, each pressure sensor 5 detects the applied pressure in real time and sends a pressure signal to the processor 4.

Step 204, the processor 4 queries and obtains a pressure event which matches the pressure signal from the memory 3, and controls the wrist watch to operate the application program corresponding to the obtained pressure event, then the procedure ends.

### Embodiment 3

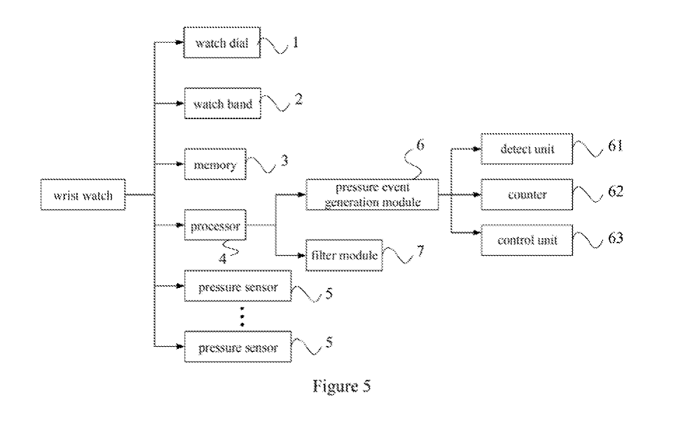

As shown in FIG. 5, in this embodiment, the processor 4 further includes a filter module 7, the difference between the present embodiment and embodiment 1 lies in: in the present embodiment, when the pressure sensor 5 is disposed on the pulse position of hand of human body, when no pressure generated from a hand action of the user is received, the pressure sensor 5 can detect pulse pressure signal generated by the pulse of human body, since the pulse of human body is regular, the pulse pressure signal also has specific change regulation, and the processor 4 thus filters the pulse pressure signal by the filter module 7 to eliminate interference to the pressure sensor 5 due to pulse of human body.

Although the specific embodiments of the present invention are illustrated as above, those skilled in the art will appreciate they are exemplary and can modify or amend these embodiments without deviating the scope and spirit of the present invention. Therefore, the protect scope of the present invention is defined by claims.

1. A wrist watch comprising a watch dial and a watch band, wherein at least one pressure sensor is provided on the watch dial and/or the watch band, and the wrist watch also comprises a memory and a processor;
the memory is configured to store at least one pressure event, at least one application program of the wrist watch and the corresponding relationship between each pressure event and an application program, and the pressure event comprises the duration time of a pressure and the range of a pressure value;
each pressure sensor is configured to detect the applied pressure in real time and send a pressure signal to the processor, and the pressure signal comprises the duration time of a pressure and a pressure value;
the processor is configured to query and obtain a pressure event which matches the pressure signal from the memory, and control the wrist watch to operate the application program corresponding to the obtained pressure event.

2. The wrist watch according to claim 1, wherein each pressure sensor has a unique identification code, and both the pressure event and the pressure signal include the identification code of the pressure sensor.

3. The wrist watch according to claim 2, wherein the processor comprises a pressure event generation module, which includes a detect unit, a counter whose initial value is zero and a control unit;
with regard to the pressure signal sent by each pressure sensor, the detect unit is configured to identify the identification code of the pressure sensor, and detect whether the duration time of a pressure exceeds a first threshold and whether the pressure value exceeds a second threshold, if the results of both the detections are “yes”, the counter is controlled to add 1 to count value, and when the count value of the counter equals to a third threshold, the control unit is activated to generate a pressure event from the pressure signal, and stores the pressure event in the memory;
the control unit is further configured to display at least one application program on the display of the wrist watch, and it is configured to pair the generated pressure event with the selected application program after receiving an instruction of selecting an application program.

4. The wrist watch according to claim 3, wherein when the pressure sensor and the processor are wired, the detect unit is configured to identify the pressure sensor according to pins on the line; when the pressure sensor and the processor are wirelessly connected, the pressure signal sent by the pressure sensor contains a field representing the identification code, the detect unit is configured to identify the pressure sensor according to the field representing the identification code.

5. The wrist watch according to claim 1, wherein when the pressure sensor locates at the pulse position of human body, the pressure sensor is configured to detect the pressure generated by the pulse of human body and send a pulse pressure signal to the processor, the processor further includes a filter module for filtering the pulse pressure signal from the pressure signal sent by the pressure sensor.

6. The wrist watch according to claim 1, wherein each pressure sensor detects the pressure generated by a hand action of human body.

7. The wrist watch according to claim 1, wherein at least one pressure sensor is disposed on backside of the watch dial and/or the watch band.

8. An operation method for application program of a wrist watch, wherein the operation method includes the following steps:
S1, the memory stores at least one pressure event, at least one application program of the wrist watch and the corresponding relationship between each pressure event and an application program, and the pressure event comprises duration time of a pressure and range of a pressure value;
S2, each pressure sensor detects the applied pressure in real time and sends a pressure signal to the processor, and the pressure signal includes duration time of a pressure and a pressure value;
S3, the processor queries and obtains a pressure event which matches the pressure signal from the memory, and controls the wrist watch to operate the application program corresponding to the obtained pressure event.

9. The operation method according to claim 8, wherein each pressure sensor has a unique identification code, and both the pressure event and the pressure signal include the identification code of the pressure sensor.

10. The operation method according to claim 9, wherein the processor comprises a pressure event generation module, which includes a detect unit, a counter whose initial value is zero and a control unit, and prior to step S1, said method further comprises a pressure event generation step;
with regard to the pressure signal sent by each pressure sensor, the detect unit identifies the identification code of the pressure sensor, and detects whether the duration time of a pressure exceeds a first threshold and whether the pressure value exceeds a second threshold, if the results of both the detections are “yes”, the counter is controlled to add 1 to count value, and when the count value of the counter equals to a third threshold, the control unit is activated to generate a pressure event from the pressure signal, and stores the pressure event in the memory;
the control unit further displays at least one application program on the display of the wrist watch, and it pairs the generated pressure event with the selected application program after receiving an instruction of selecting an application.

11. The operation method according to claim 10, wherein, in the pressure event generation step, when the pressure sensor and the processor are wired, the detect unit is configured to identify the pressure sensor according to pins on the line; when the pressure sensor and the processor are wirelessly connected, the pressure signal sent by the pressure sensor contains a field representing the identification code, the detect unit is configured to identify the pressure sensor according to the field representing the identification code.

12. The operation method according to claim 8, wherein, in the operation method, when the pressure sensor locates at the pulse position of human body, the pressure sensor detects the pressure generated by the pulse of human body and sends a pulse pressure signal to the processor, and the processor further includes a filter module for filtering the pulse pressure signal from the pressure signal sent by the pressure sensor.

13. The operation method according to claim 8, wherein, in step S2, each pressure sensor detects the pressure generated by a hand action of human body.

14. The wrist watch according to claim 2, wherein when the pressure sensor locates at the pulse position of human body, the pressure sensor is configured to detect the pressure generated by the pulse of human body and send a pulse pressure signal to the processor, the processor further includes a filter module for filtering the pulse pressure signal from the pressure signal sent by the pressure sensor.

15. The wrist watch according to claim 3, wherein when the pressure sensor locates at the pulse position of human body, the pressure sensor is configured to detect the pressure generated by the pulse of human body and send a pulse pressure signal to the processor, the processor further includes a filter module for filtering the pulse pressure signal from the pressure signal sent by the pressure sensor.

16. The wrist watch according to claim 4, wherein when the pressure sensor locates at the pulse position of human body, the pressure sensor is configured to detect the pressure generated by the pulse of human body and send a pulse pressure signal to the processor, the processor further includes a filter module for filtering the pulse pressure signal from the pressure signal sent by the pressure sensor.

17. The operation method according to claim 9, wherein, in the operation method, when the pressure sensor locates at the pulse position of human body, the pressure sensor detects the pressure generated by the pulse of human body and sends a pulse pressure signal to the processor, and the processor further includes a filter module for filtering the pulse pressure signal from the pressure signal sent by the pressure sensor.

18. The operation method according to claim 10, wherein, in the operation method, when the pressure sensor locates at the pulse position of human body, the pressure sensor detects the pressure generated by the pulse of human body and sends a pulse pressure signal to the processor, and the processor further includes a filter module for filtering the pulse pressure signal from the pressure signal sent by the pressure sensor.

19. The operation method according to claim 11, wherein, in the operation method, when the pressure sensor locates at the pulse position of human body, the pressure sensor detects the pressure generated by the pulse of human body and sends a pulse pressure signal to the processor, and the processor further includes a filter module for filtering the pulse pressure signal from the pressure signal sent by the pressure sensor.
